(19) United States
(12) Patent Application Publication
(10) Pub. No.: US 2010/0242155 A1
(43) Pub. Date: Sep. 30, 2010

(54) Headgear equipped with laser hair care apparatus

(76) Inventor: John F. Carullo, JR., Royse City, TX (US)

Correspondence Address: Handley Law Firm, PLLC;Roger N. Chauza, PC, PO BOX 140036, IRVING, TX, 75014, US

(21) Appl. No.: 12/586,693

(22) Filed: Sep. 25, 2009

Related U.S. Application Data
(60) Provisional application No. 61/210,957, filed on Mar. 25, 2009.

(51) Int. Cl. A42C 99/00; A61N 5/06

(52) U.S. Cl. 2/171.2; 607/89

(57) ABSTRACT

Hair treatment headgear having the appearance of common caps, dress hats, casual hats, etc. The headgear can be a baseball cap having a crown portion equipped with plural laser diodes for illuminating the hair of the person wearing the cap. The cap is also equipped with a battery and a switch to control the time in which the laser diodes are activated. The person's hair can thus be treated in a hands-free manner while wearing the baseball cap, where such treatment remains inconspicuous and unknown to others.

### RELATED APPLICATION

This US non-provisional patent application claims the benefit of pending U.S. provisional patent application Ser. No. 61/210,957 filed Mar. 25, 2009, and entitled “Headgear Equipped With Laser Hair Care Apparatus.”

### TECHNICAL FIELD OF THE INVENTION

The present invention relates in general to laser hair care products, and more particularly to head gear equipped with light emitting devices, and more specifically laser diodes.

### BACKGROUND OF THE INVENTION

Studies and experience have shown that the growth of a person's hair can be stimulated by the use of light emitted from laser diodes and the like. It is believed that the stimulation of hair growth is better achieved using laser light than lotions, gels and other similar products. The application of laser light of a particular wavelength and power is generally administered in a clinical setting, such as an office where appointments are made by the persons desiring the stimulation of hair growth. While not limited to males, females also experience a thinning of the hair on the scalp, and thus receive the benefit from the use of hair growth stimulation products.

It is customary for hair treatment clinics, spas, salons, and the like, to use equipment to administer laser light to the customer. The equipment generally includes a seat or chair in which the customer can comfortably sit for the time in which the laser light is applied to the customer's head or other area to be treated. The treatment equipment includes a movable head cover or hood which can be moved by mechanical means over and into close proximity with the customer's scalp. Embedded in the hood are a number of laser devices, such as 50-110 laser light emitting diodes that each emit a beam of light. When in close proximity to the scalp, the laser diodes, or other light emitting devices, are activated for a prescribed period of time to stimulate the growth of the hair follicles. The success of the treatment requires a number of sessions in which the customer must make an appointment and spend no less than a half hour, or so, during the treatment session. A typical clinical protocol is two times per week for twenty-six weeks and then once per week for about twenty-six weeks. An annual program can cost in the neighborhood of $3,950.

There are other hair treatment devices that stimulate hair growth. One such device is a hand-held hair brush instrument with laser diodes fixed thereto. See U.S. Pat. No. 7,258,695 by Carullo, Jr., et al., which discloses a hair restoration device that can be used to brush a person's hair, and at the same time the light emitted from the laser diodes bathes the roots of the hair with light that restores hair growth. Although this brush is effective to stimulate the growth of a person's hair, the user must continue the treatment for about fifteen minutes for effective results. This not only requires one to continuously brush the hair for the specified time period, but prevents the person from conducting other activities. Since the treatment is for fifteen minutes, or so, the person's arm must be held up for such time. This leads to tiring of a person's arm and often results in a shortened period of treatment.

From the foregoing, it can be seen that a need exists for a hair stimulation product which can be used by the person desiring the same, without having to go to a treatment clinic or office. A further need exists for a hair stimulation product which can be administered and used by the customer at home, outside or other places without interrupting the normal schedule of the person. Yet another need exists for a hair stimulation product that resembles a normal headgear, such as a baseball cap, hat, etc., with the light emitting or laser devices incorporated into the headgear so that when worn and activated, the user's hair is stimulated during wearing of the headgear. A further need exists to allow for more practical hands-free treatment other than the arduous task of brushing ones hair while standing in a stationary position. Another need exists for a hair care treatment device that is administered by the user and is thus more cost effective and flexible in that no office visits or appointments are necessary.

### SUMMARY OF THE INVENTION

In accordance with the principles and concepts of the invention, there is disclosed a hair care product in the nature of a baseball cap or a casual or dress hat having light emitting or laser diodes embedded inside the headgear so that when activated, the appropriate portion of the user's scalp is illuminated all at once and hair growth is correspondingly stimulated. The headgear can be worn whenever desired and requires no further attention during the treatment. Thus, the person wearing the headgear where the laser light emitting diodes have been activated, can carry on other routine duties without interruption and without being mindful of the administration of the treatment during the session. The headgear of the invention can be used at home and is hands-free, thus allowing the user to conduct other activities at any place. The user of the headgear can drive a vehicle and can conduct other activities that require a high degree of attention. The treatment of hair using the headgear of the invention is also less expensive and less time consuming than the corresponding service provided by clinics, salons and the like.

According to another feature of the invention, the headgear includes a switch and a battery for powering the light emitting devices. The user can switch the power to the light emitting devices to at any time a treatment is desired. The battery and the switch can be incorporated into the head gear so as to be readily available, but generally inconspicuous to others.

According to yet another feature of the invention, the light emitting devices can be arranged within the headgear so as to be located to provide a therapeutic light at areas on the person's scalp where hair treatment is desired. To that end, the light emitting devices can be arranged in headgear in the same shape as the area of baldness or thinning experienced by the user.

With regard to yet another embodiment of the invention, the hair treatment device can be a dome-shaped member with light emitting devices embedded therein. The dome-shaped member can be inserted into a headgear such as a baseball cap or hat, to thereby provide treatment while wearing the headgear.

In accordance with another embodiment of the invention, disclosed is a headgear for administering hair restoration treatments. The apparatus includes a headgear adapted for wearing by a person, where the headgear is constructed to appear as well known headgear worn by persons in public. Included is one or more light emitting devices attached to the headgear to illuminate a person's head when wearing the headgear. A battery is included for powering the light emitting devices. The battery is attached to the headgear, whereby the headgear can be worn anywhere while administering the light to the wearer's head without the treatment being conspicuous.

According to a further embodiment of the invention, disclosed is a headgear for administering hair restoration treatments, where the headgear comprises a baseball cap having a crown portion and a bill attached to the crown portion. One or more light emitting devices are attached within the crown portion of the baseball cap to illuminate a person's head when wearing the baseball cap. A battery powers the light emitting devices. The battery is attached to the baseball cap, whereby the baseball cap can be worn anywhere while administering the light to the wearer's head without the treatment being conspicuous.

In accordance with yet another embodiment of the invention, disclosed is a method of treating a person's hair with light energy. The method includes arranging a plurality of light emitting devices in a headgear to be worn by the person, constructing the headgear so that it can be placed on the person and the person can wear the headgear and move about outdoors with the headgear, and fastening a switch and a battery to the headgear, where the switch is actuated by the person wearing the headgear. The switch is adapted for controlling when power from the battery is applied to the light emitting devices.

### BRIEF DESCRIPTION OF THE DRAWINGS

Further features and advantages will become apparent from the following and more particular description of the preferred and other embodiments of the invention, as illustrated in the accompanying drawings in which like reference characters generally refer to the same parts, functions or elements throughout the views, and in which:

FIG. 1 is a bottom view of a hair care cap constructed according to one embodiment of the invention;

FIG. 2 is a bottom view of a hair care cap constructed according to a second embodiment of the invention;

FIG. 3 is a bottom view of a hair care cap constructed according to a third embodiment of the invention;

FIG. 4 is a bottom view of a hair care cap constructed according to a fourth embodiment of the invention;

FIG. 5 is a bottom view of a dome-shaped liner equipped with a number of laser diodes;

FIG. 6 is a bottom view of a rigid basket used to support a laser diode member in a cap;

FIG. 7 is a laser diode member constructed to be attached to the basket of FIG. 6;

FIG. 8 is a self-contained laser diode member adapted for insertion into a cap;

FIG. 9a is a bottom view of a light emitting insert, for use within a headgear;

FIG. 9b is a side view of the light emitting insert of FIG. 9a;

FIG. 10 is a side view of a baseball cap incorporating the insert of FIGS. 9a and 9b;

FIG. 11 is a side view of a baseball cap incorporating the light emitting devices, with optional electrical cord connections thereto; and

FIG. 12 is a bottom view of another embodiment of the light emitting headgear of FIG. 9a.

### DETAILED DESCRIPTION OF THE INVENTION

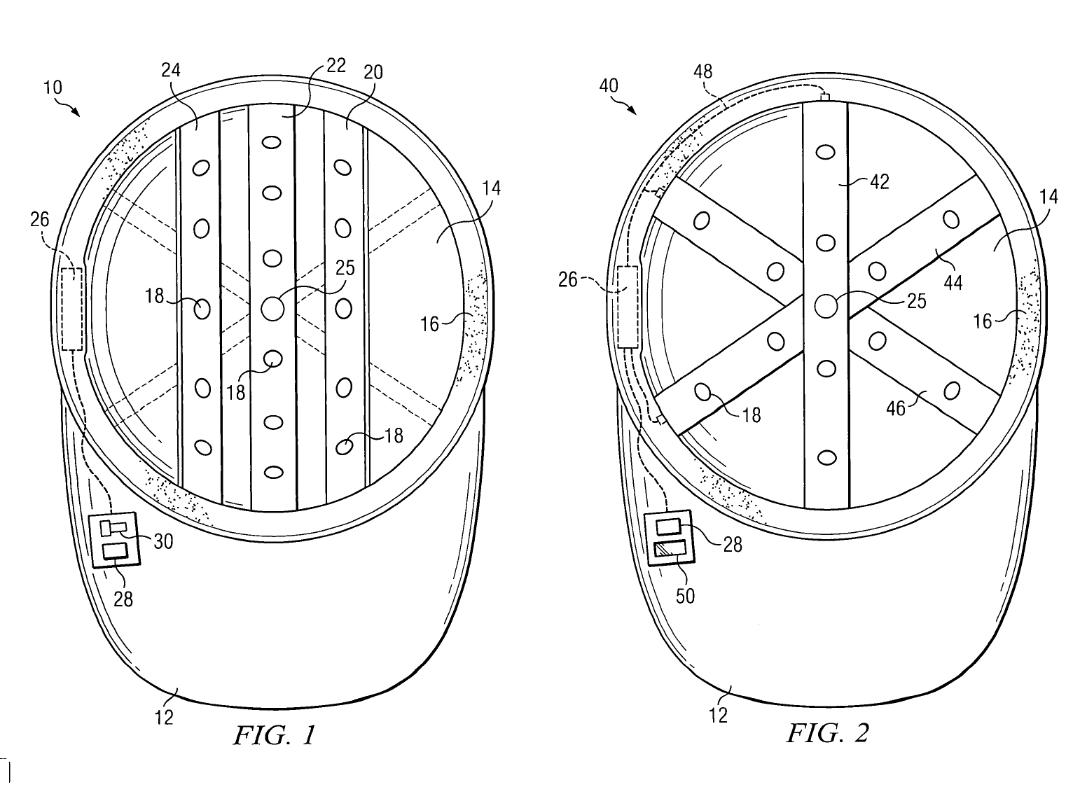

With reference to FIG. 1, there is shown a baseball cap 10 constructed to provide hair growth stimulation by light emitting devices. The view of FIG. 1 is from the bottom of the cap 10, which illustrates the inside of the crown 14 of the cap 10. The cap 10 is of conventional construction having a rather rigid visor or bill 12 to shade the wearer's eyes from the sun. The cap 10 also includes the crown portion 14 that is generally hemispherical-shaped to cover the top portion of a person's head. As is conventional, the bill 12 is attached to the crown 14, and the circular edge of the crown 14 includes a sweat band 16 that engages the person's head. Often, the sweat band 16 portion of the cap 10 is constructed so as to be expandable (not shown) so that the cap 10 can fit a number of different head sizes. In order to form the dome-shaped crown 14, the crown 14 of the cap 10 is constructed with a number of pie-shaped panels that are sewn together at respective seams.

In accordance with an important feature of the invention, the baseball cap 10 is equipped with a number of light emitting devices, one shown as numeral 18. In one embodiment of the invention, the light emitting devices 18 are laser diodes adapted for emitting light in the spectrum of about 400-1020 nm and more specifically about 650 nm, at a power in the range of about twenty-five mw, or less, and preferably about 5 mw or less. Typically, the laser diodes 18 are two terminal discrete devices powered by a DC voltage source of about 3.7 volts, or other suitable voltage.

As can be seen from FIG. 1, there are three strips 20, 22 and 24 that extend from the front of the crown 14 to the back of the crown 14. This arrangement of the strips 20-24 in the crown 14 is adapted for use by males who experience thinning of hair or baldness from the forehead over the crown of the head to the back of the head. Other arrangements of light emitting diodes 18 and strips 20-24 can be employed to treat other areas of the person's head. The strips 20-24 can be constructed of rigid strips of material, such as plastic, fabric, stiff paper, etc. In the embodiment of the baseball cap 10 shown, the strips 20-24 are constructed of a Mylar® material or plastic, each having a width of about 0.5-1.0 inches and a thickness of about 0.25 inches. Many other widths and thicknesses of the strips can be utilized. The ends of each strip 20-24 are anchored between the material of the crown 14 and the sweat band 16. The ends of the strips 20-24 can be adhered between the crown 14 and the sweat band 16, sewn, or fastened by any other suitable means. The center strip 22 can be supported to the crown 14 with a pin 25 which extends through the crown 14 and terminates on top of the crown 14 of the cap 10 with a standard top button. The midsections of the strips 20-24 can also be bonded to the underside of the crown 14 material of the cap 10 to maintain the strips 20-24 in place and prevented from movement during adjustment of the cap 10 on the person's head. An adhesive, Velcro® type material or other suitable attachment means can be used.

The plastic strip 20 is illustrated as having five laser diodes 18 attached thereto, the middle strip 22 has six laser diodes 18 attached thereto, and the other strip 24 has five laser diodes 18 attached thereto. The strips can have more or fewer light emitting devices than shown. The strips 20-24 can be fabricated to include holes therein, through which a respective laser diode 18 can be inserted and fixed therein, such as by a bonding agent. The lens or output of each laser diode 18 is exposed toward the person's head, and the two metal terminals of each laser diode 18 is on the other side of the strip, adjacent to the crown material 14 of the cap 10. The laser diodes 18 are each electrically connected together in parallel using two conductors (not shown). The conductors can be bonded to the backsides of the respective strips 20-24. Alternatively, the strips 20-24 can be constructed of a synthetic film having the conductors embedded therein, much like electrical conductor flex strips used in many types of electronic machines. The ends of the conductors can be connected with other conductors (not shown) between a rechargeable battery 26 and a manually-operated switch 28 which is mounted to the bill 12 of the cap 10.

The battery 26 can be of any conventional rechargeable type providing about 3.7 volts. Lithium rechargeable batteries are believed to be well adapted for use with the various embodiments of the invention. Indeed, the battery 26 can be made up of more than a single wafer-type battery that is coupled either in parallel or series to provide the voltage and power required by the laser diodes 18. The battery 26 can be fastened between the sweat band 16 and the crown 14 of the cap 10, or in the front of the cap area. The sweat band 16 can be formed with a pocket to hold the battery 26. Alternatively, a patch of material can be sewn to the inside or outside surface of the of the crown 14 to form a pocket to hold the battery 26 therein. The connector or socket that allows the battery to be recharged can be located on or behind the sweat band 16, or attached to the bill of 12 the cap 10. The connector or socket can also be attached to the pocket. An adapter can be employed to connect to the socket area to allow for charging of the battery 26. The switch 28 can be mounted either on the top or bottom surface of the bill 12 so as to be accessible by the user of the cap 10. Moreover, a timer 30 can be connected in the circuit with the switch 28 to allow the laser diodes 18 to be actuated for only a specified period of time. In this manner, the optimum results can be obtained and the user can be assured that his/her scalp will not be overexposed (or underexposed) to the energy of the laser diodes 18. The timer 30 can be a standard monostable circuit that is triggered by activation of the switch 28, and is active for a predetermined period of time, after which the monostable timer 30 automatically returns to the off state, thereby removing power from the laser diodes 18. The monostable timer circuit 30 can be made as a flat module that also houses the battery 26 and is mounted to the crown portion or other area of the cap 10. When used with the timer 30, the switch 28 would preferably be a miniature momentary push button type of switch. Otherwise, without the use of a timer 30, the switch 28 would be a slide or toggle type or other type of switch that would require activation to allow the battery current to power the laser diodes 18, and would be again actuated to remove power from the laser diodes 18. The switch 28 could be a miniature type embedded in the material of the bill 12 so that it can be actuated by simply squeezing a specific location in the bill 12 where the switch is located. In this manner, the switch 28 is not visible, but is only a small bump or bubble in the bill 12. The switch 28 can be located at other locations in the cap 10, including the sweat band 16. With the timing circuit 30 mounted with the battery 26, there can be a display mounted adjacent the switch 28 to provide a visual indication of the time remaining for the treatment. Alternatively, or in addition thereto, a small electrically operated buzzer can be used.

The laser hair care cap 10 can be constructed starting with a standard baseball cap, casual or dress hat, and adding the various components thereto. In other words, the headgear is not substantially different from a standard baseball cap or hat, but is additionally fitted with the laser diodes 18, the battery 26 and switch 28 to provide the therapeutic effects noted above. In a preferred embodiment, the headgear comprises a baseball cap 10.

The hair stimulation cap 10 can be used in the following manner. As can be appreciated, from outward appearance the cap 10 does not look much different from a conventional baseball cap. During periods in which the therapeutic effects of the cap 10 are not desired, then the person can simply wear the cap 10 in the normal manner. When it is desired to activate the cap 10 to illuminate the person's scalp with the laser light and stimulate the growth of hair, then the person need only activate the switch 28, whereupon the laser diodes 18 emit light of the proper wavelength to stimulate hair growth. As the person wears the cap 10 with the laser lights 18 activated, the light illuminates the person's scalp at the proper locations to irradiate the hair roots and follicles. Importantly, the laser diodes 18, being fixed to the inner surface of the crown 14 of the cap 10, are spaced from the person's scalp a distance to allow the beams of laser light to diffuse outwardly and effectively cover the areas of the scalp without leaving any untreated areas, except on the sides of the person's scalp. However, as noted above, the placement of the laser diodes 18 can be modified to achieve the desired area of coverage of the laser light that illuminates the person's head. Importantly, the treatment can be achieved at any time without having to go to a clinic, and without having to repeatedly brush ones hair with a hair treatment brush. Thus, the treatment using the laser hair care cap 10 can be repeated as often as practical without having to change one's schedule or work habits significantly. When treating the scalp with the laser light, the cap 10 is worn in the usual manner and other people in the immediate area will not be aware of the treatment.

In order to provide a headgear that can selectively illuminate desired areas of a person's head, the different laser diodes 18 can be connected together in different switchable groups. In other words, the laser diodes 18 in the back of the cap 10 adjacent to the back of a person's head can be connected together through a switch to the battery 26. Similarly, the group of laser diodes 18 in the front of the cap 10 can be connected together through another switch to the battery 26. Other groups of laser diodes 18 and corresponding switches can be utilized. One or both of the switches can be switched on selectively to cause illumination of the specified areas of the person's head. Thus, for a person who is experiencing thinning of hair in a particular area of the scalp, the switch that controls the laser diodes 18 of that area can be switched on to illuminate only that area. The battery power is thus conserved. In view that the thinning of a person's hair typically occurs in three or four general patterns, the laser diodes 18 can be grouped together to cover those particular patterns. Indeed, one or more of the laser diodes can be in different groups. The switches can be miniature switches located in the bill 12. or other location of the cap 10.

With reference to FIG. 2, there is illustrated a hair care cap 40 constructed according to another embodiment of the invention. In this embodiment of the invention, the cap 40 has incorporated into the crown 14 three strips 42, 44 and 46 that have laser diodes, one shown as numeral 18, mounted thereto in the manner described above. However, in this embodiment, the strips 42-46 are not parallel, but are arranged like the spokes in a wheel, namely, they extend radially outwardly from the center pin 25, to the sweat band 16. With this arrangement, the coverage of the laser light on the person's head is wider, thereby covering more of the area of the person's hair to be rejuvenated. As can be appreciated, more or fewer laser diodes can be employed than shown. More laser diodes 18 would be used to increase the magnitude of the laser light that illuminates the person's head. One wire would be extended from the battery 26 to power the array of laser diodes 18, and another wire would be extended from the battery 26 as ground or common to the array of laser diodes 18. A wire is shown as numeral 48 connected to strip 42 In this embodiment, a visual display 50 is shown mounted to the bottom side of the cap bill 12, together with the switch 28. The display 50 functions to provide a visual indication of the treatment time that has elapsed.

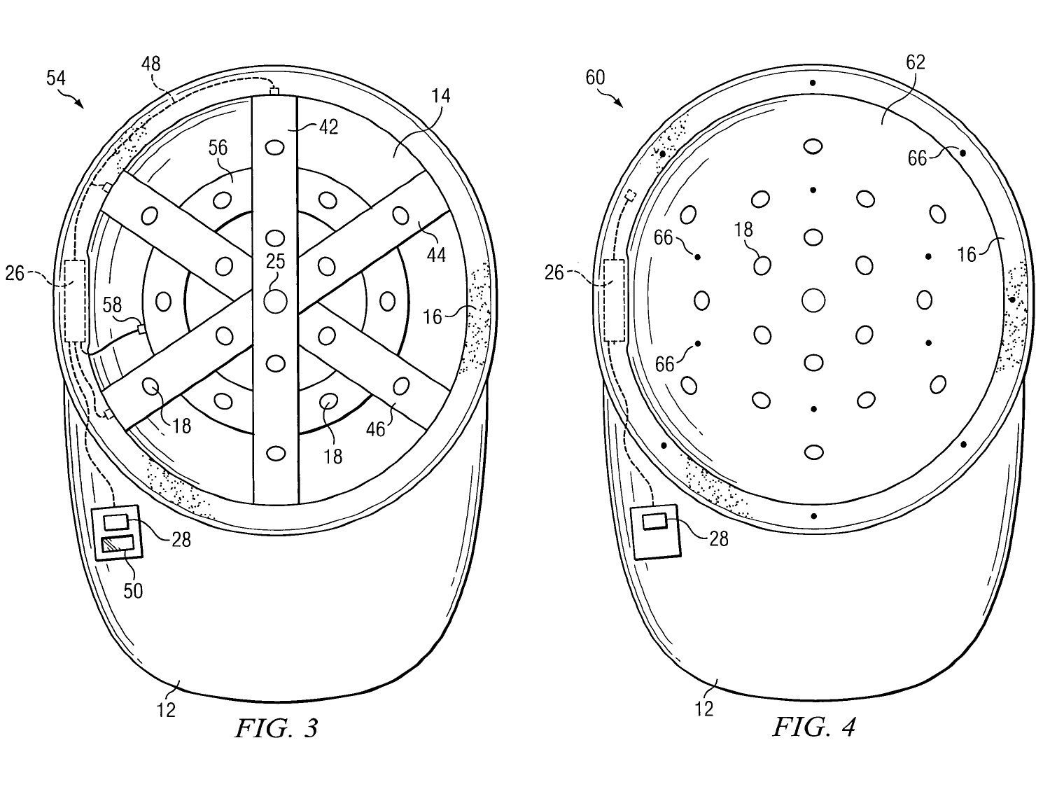

FIG. 3 illustrates yet another embodiment according to the invention. Here, the cap 54 is substantially similar to that shown in FIG. 2, but additionally includes a circular strip 56 centered about the crown pin 25. The location of the circular strip 56 is similar to a latitude line around the earth. The function of the circular strip 56 is to allow placement of additional laser diodes 18 between the radial strips 42, 44 and 46. The additional set of laser diodes 18 on the circular strip 56 increases the concentration of the laser light on the top portion of a person's head. An electrical connection to the laser lights 18 on the circular strip is shown at numeral 58.

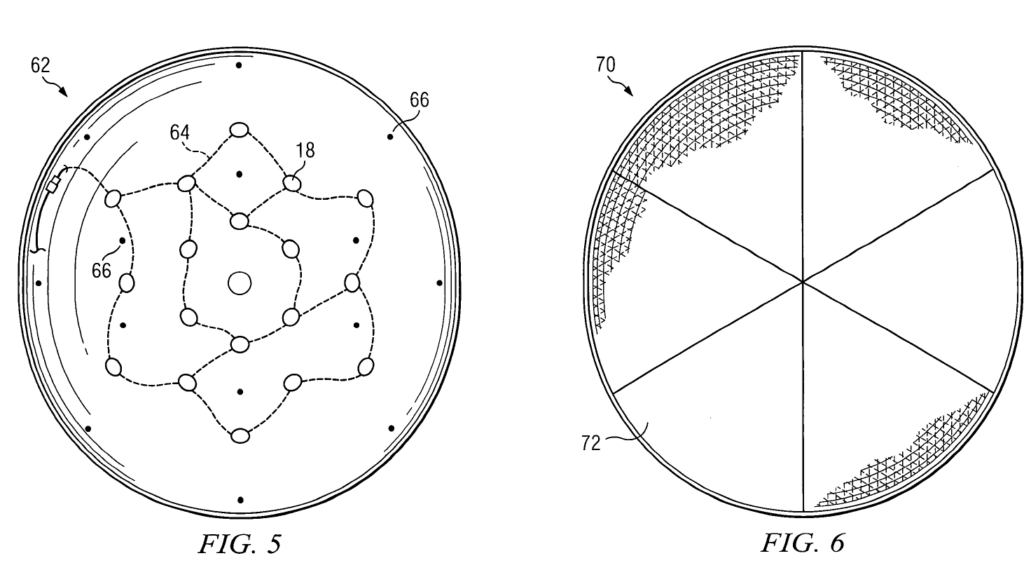

FIGS. 4 and 5 depict a hair care cap 60 employing a dome-shaped liner 62 that is adapted for insertion into a baseball cap 60, or in any other type of cap or head covering. The liner 62 is constructed for mounting thereto a plurality of laser diodes, one shown as 18. The liner 62 is preferably constructed from a molded piece of thin and flexible Mylar or plastic material, with holes for bonding therein of the laser diodes 18. The dome 62 can be perforated with other holes (not shown) to allow for air ventilation. Materials other than Mylar can be used, including rubber, plastic, etc. The liner 62 is preferably installed into the baseball cap 60 by turning down the sweat band 16, inserting the liner 62 into the crown 14 of the cap 60, wiring the battery 26 to the conductors that extend to the laser diodes 18, and then returning the seat band 16 to its original position. Again, any number of mechanisms can be used to fix the liner 62 in the cap 40, including glue, sewing, Velcro® strips, snaps, etc.

The laser diodes 18 are wired together as shown by the wires 64 (broken lines) in FIG. 5. A small two-terminal electrical connector can be fastened to the liner 62 and connected to a corresponding electrical connector associated with the battery 26. This allows easy replacement or cleaning of the liner 62. The liner 62 can be attached to the underside side of the crown 14 of the cap 60 by bonding the same at points 66, shown by dots, or adhered in other suitable ways. With this arrangement, laser diodes 18 can be mounted to the dome 62 at any and all locations to treat any area of a person's scalp.

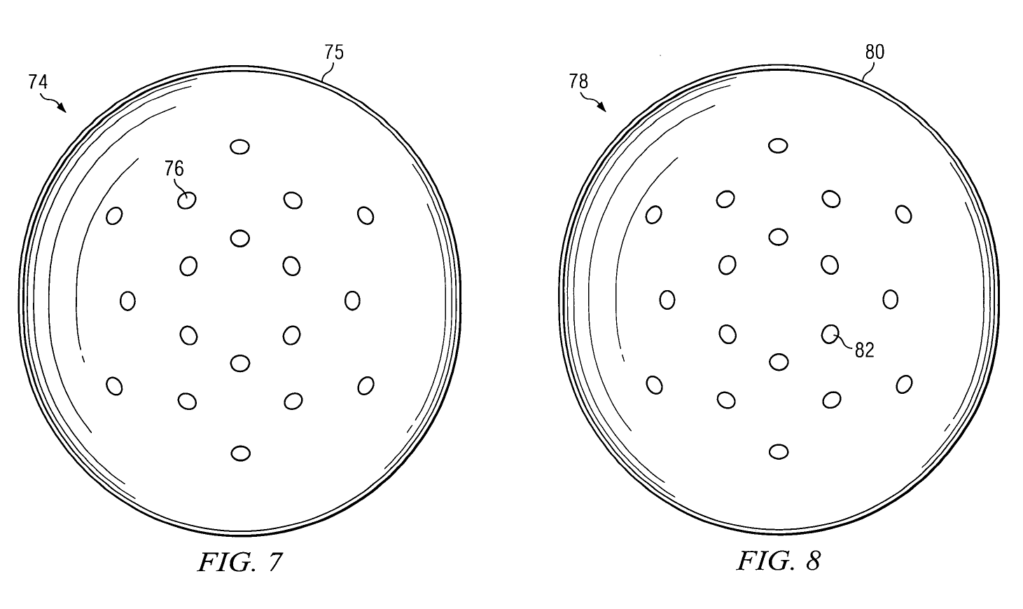

FIGS. 6 and 7 illustrate a hair care cap structure constructed according to another embodiment of the invention. FIG. 6 illustrates a headgear comprising a metal frame or basket 70 constructed of a thin metal layer or a screen that is dome shaped. The frame can be shaped like a wheel with spokes and the rim. The metal dome 70 can be constructed of plural triangular-shaped sections, one shown as numeral 72, although the basket 70 could be made as a unitary structure. The basket 70 functions as a frame for attachment thereto of the light emitting member 74 depicted in FIG. 7.

FIG. 7 illustrates the light emitting member 74 constructed as a thin plastic or Mylar dome 75 to which a number of light emitting devices 76 are attached. The light emitting member 74 is fabricated to fit within the basket 70 of FIG. 6. The electrical conductors of the light emitting member 74 can be embedded in or on the dome material. Of course, the light emitting member 74 can be constructed of other materials, including synthetic and natural materials.

The battery and switch employed to control the light emitting member 74 are attached to the cap or hat into which the light emitting member is held. Conductor wires and/or a connector can be used to provide electrical connections between the battery and switch and the light emitting devices 76 of the light emitting member 74.

The light emitting member 74 is attached to the basket 70 by a bonding agent, adhesive or other suitable means to make a unitary structure. The composite structure of FIG. 6 and FIG. 7 can be incorporated into a standard baseball cap and used in the manner described above to stimulate the growth of hair. In other words, the unitary hair treatment structure can be marketed separately so that users can use their own caps during the treatment sessions. Indeed, the unitary structure can be installed in a cap by folding the sweat band 16 down, inserting the unitary structure into the underside of the cap, and then returning the sweat band 16 to its original position to hold the peripheral edge of the unitary structure in the cap. Advantageously, the unitary structure can be placed in one cap for a hair treatment on one day, and placed in another cap the next day for a subsequent treatment. In order to provide optimum laser light coverage of a typical area of hair loss for a male, the pattern of laser diodes can be adapted to provide an hour glass shaped pattern to match the area of hair loss.

FIG. 8 illustrates a self-contained laser diode head cover 78 that is adapted for insertion into a conventional cap. The head cover 78 is constructed with a rigid or semi-rigid material, such as a thin film of Mylar material or plastic. The head cover 78 includes a plastic base member 80 that is molded in a dome shape, or other shape adapted for fitting into a cap or other headgear. The dome-shaped base member 80 includes holes therein for capturing and holding respective light emitting devices therein, such as laser diodes, one shown as numeral 82. The laser diodes 82 can be press fit into the holes, or glued in the holes of the base member 80. The laser diodes 82 are oriented to direct the light toward the internal portion of the dome-shaped base member 80. Moreover, the base member 80 can be equipped with electrical sockets into which the laser diodes 82 can be inserted to automatically connect the terminals of the laser diodes 82 with the terminals of the socket. The conductors for coupling the battery current to all of the laser diodes 82 can be embedded in the base member 80 and connected to each terminal of the respective sockets. In this manner, a laser diode 82 can be easily replaced if it becomes defective. The hair treatment structure can be marketed separately so that users can use their own caps during the treatment sessions.

The switch and battery used by the base member 80 are attached to the cap or hat into which the base member 80 is held. Conductor wires and/or a connector can be used to provide electrical connections between the battery and switch and the light emitting devices 82 of the base member 80.

The laser head cover 78 can be inserted and maintained in a cap in the same manner described above in connection with the embodiment of FIGS. 6 and 7. In addition, the laser diode head cover 78 can be attached the inner surface of the crown of a cap by glue, by sewing the same, snaps, Velcro® strips, or other suitable means.

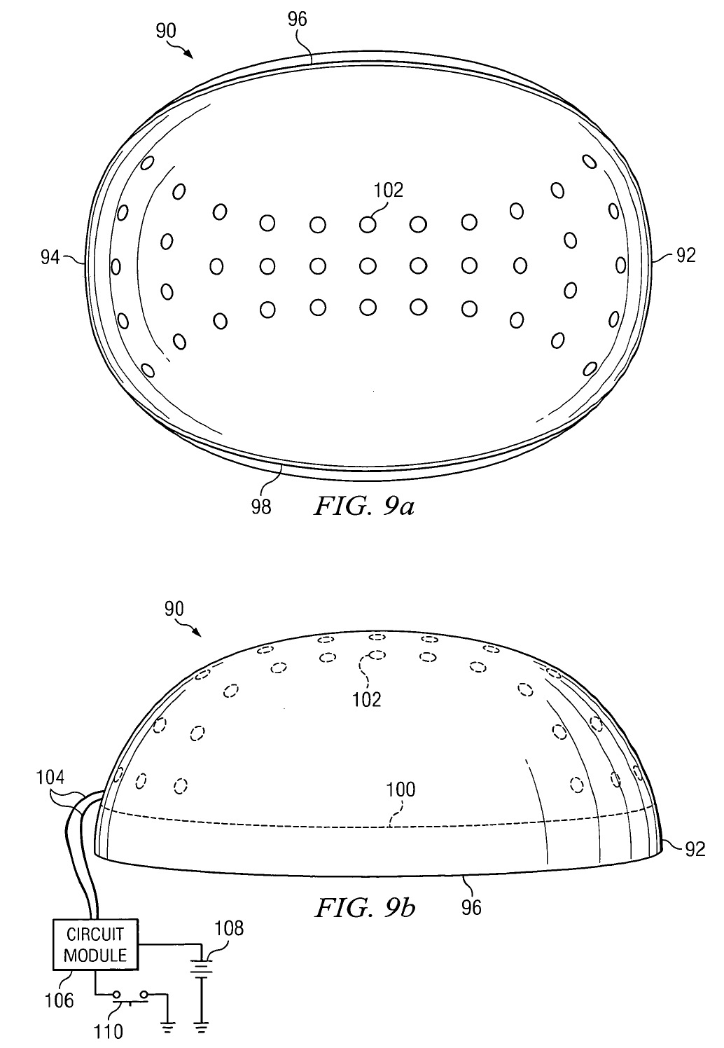

FIGS. 9a and 9b illustrate another embodiment of a light emitting insert 90 that can be inserted into a headgear. The light emitting insert 90 is adapted for insertion into a baseball type cap, although the light emitting insert 90 can be adapted for use with many other types of headgear. For ease of installation, the light emitting insert 90 is adapted for insertion within the crown portion of a baseball cap and captured therein by the sweat band. The insert 90 is well adapted for fitting into a baseball type cap that is constructed with a complete sweat band encircling the entire edge of the cap. In other words, the light emitting insert 90 is shaped to fit within the baseball cap, and is shaped to surround at least a portion of the person' head. The peripheral edge of the light emitting insert 90 includes a frontal peripheral edge 92 that is captured between the bottom of the crown and the sweat band portion in the front of the cap. The insert 90 is also constructed with a back peripheral edge 94 that is captured between the crown portion and the sweat band at the back of the cap. Side peripheral edges 96 and 98 are provided for insertion into the baseball cap and captured between the side crown portions and the sweat band at the respective sides of the cap. When installing the light emitting insert 90, the sweat band of the baseball cap is turned down, the light emitting insert 90 is inserted into the crown portion of the baseball cap, and then the sweat band is turned back up to its original position to capture the insert 90 within the baseball cap. The portion of the peripheral edge of the light emitting insert 90 that is captured behind the sweat band is shown by the broken line 100. For baseball caps that have a mechanism to adjust the size thereof, the insert 90 can be constructed with a wide notch in the back edge to allow easy adjustment of the size of the cap.

The light emitting insert 90 is constructed of a plastic sheet, Mylar, or other suitable material that holds its shape while worn. The shape of the insert 90 is molded so as to conform to the inside of the baseball cap, or other headgear employed, as well as shaped to fit comfortably over the person's head, it being realized that the general shape of a person's head is not hemispherical. Rather, a person's head is generally narrower between the ears, as compared to the front to back dimension. FIG. 9a illustrates the shape of the light emitting insert 90 which is narrower between the sides 96 and 98, as compared to the distance between the front 92 and back 94 of the insert 90.

While the light emitting devices, one shown as numeral 102, can be placed anywhere on the insert 90, the pattern shown is that generally of an hour glass. This pattern baldness in men is prevalent. The light emitting devices 102 can be laser diodes, LEDs or other suitable devices that emit light in the wavelength and power sufficient to stimulate the hair follicles. Preferably, the light emitting insert 90 is constructed of a moldable synthetic material in the same manner as many flexible electrical cables or flexible circuit boards. In other words, the conductors that connect all of the light emitting devices 102 together are embedded in the plastic material and extend throughout the insert to locations where the light emitting devices 102 are located. The two conductors for each light emitting device 102 terminate at two terminals where the light emitting device is electrically connected. Each light emitting device 102 can be bonded to the material of the insert 90. The light emitting devices 102 are preferably connected in parallel so that the full power of the battery is applied to each such device 102. The lens of each light emitting device is directed to the inner portion of the insert so as to illuminate the person's head when the baseball cap equipped with the insert 90 is worn. While not shown, the insert 90 can include a number of ventilation holes to provide circulation of air through the insert 90.

The wires 104 connected to the conductors embedded in the light emitting insert 90 exit the insert 90 and can be connected to a circuit module 106. A connector (not shown) can be utilized for easy connection of the circuit module 106 to the conductors of the insert 90. The circuit module 106 can be a timer to provide a specified period of time in which the light emitting devices 102 are activated once the switch 110 is operated. A battery 108 is connected to the timer circuit 106 to provide power thereto as well as to the light emitting devices 102. Alternatively, the timer 106 can be omitted, whereupon the switch 110 can be activated to the on position to operate the light emitting devices, and thereafter activated to remove power from the light emitting devices 102. The circuit timer 106 can also be molded within the insert 90 together with the conductors embedded therein.

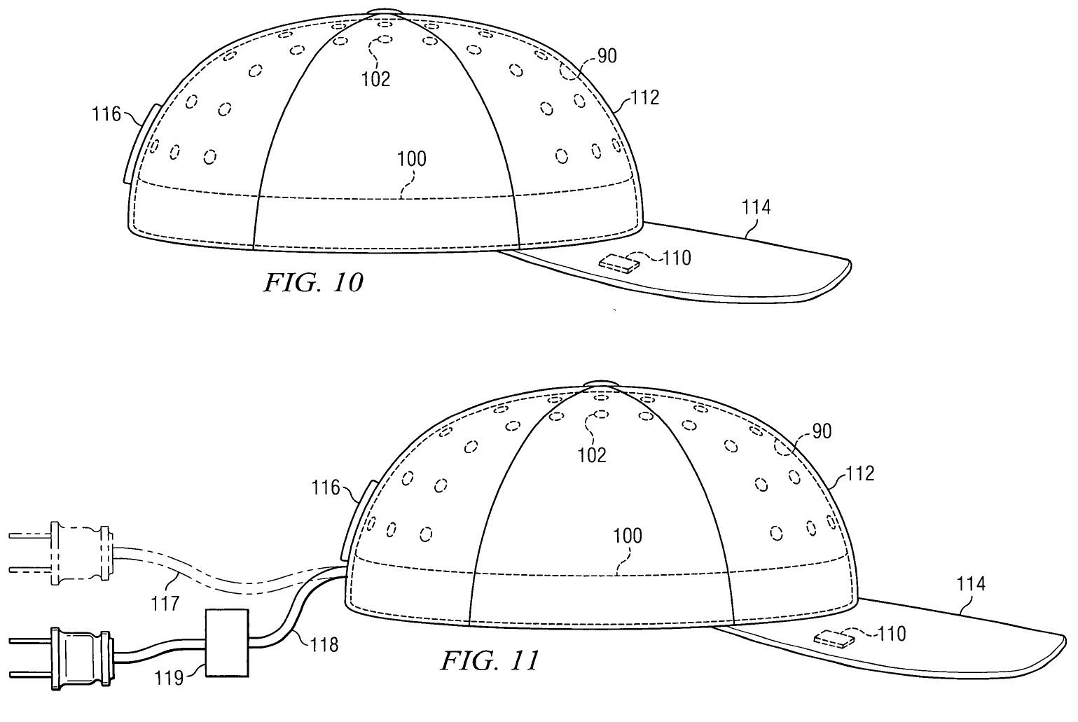

FIG. 10 illustrates the light emitting insert 90 inserted and captured within a baseball type cap 112 of the type having a bill 114. The sweat band is turned up in its normal position with the edge 100 shown in broken line, and with the peripheral edge of the insert 90 captured between the sweat band and the bottom portion of the crown of the cap 112. As can be appreciated, the light emitting insert 90 is not visibly discernible when worn by the user.

The baseball cap 112 can be constructed with a pocket or pouch 116 to hold the battery 108 therein. The pocket 116 can be constructed with a flap (not shown) that snaps or otherwise prevents inadvertent displacement of the battery 108. The wires 104 exiting the insert 90 can be routed to the battery pocket 116 and connected to contacts fastened in the pocket 116. Thus, the battery 108 can be easily replaced when no longer able to hold a charge. The pocket 116 can include a connector (not shown) for electrically connecting a battery charger to the battery 108 so that the charge in the battery 108 can be replenished. The switch 110 can be fastened to the underside of the bill 114 of the cap 112 so as not to be highly visible. The user can thus easily squeeze the switch 110 and thus activate the circuit module 106, if provided, or the light emitting devices 102 directly. The wires 104 can be routed to the pocket 116 from the insert 90 through a small hole in the material of the cap 112. Similarly, wires can exit the insert 90 from the front thereof to the area in the front of the cap 112 and connect to the switch 110 in the bill 114. Alternatively, wires exiting the insert 90 can be routed around the sweat band to appropriate locations of the cap 112 to provide electrical connections to all of the circuits.

In the event that the headgear is worn at locations where it is desired to use AC electrical current, the headgear, such as the baseball cap 112 of FIG. 11, can be equipped with an AC cord 117 that can be plugged into an AC outlet providing the standard AC voltages. The cord 117 can be of the coiled type or any other conventional AC cord. The cap 112 can be equipped with a converter circuits for converting the AC voltage to the desired DC voltage. The DC voltage can be used to power the light emitting devices 102 directly, or used to recharge the battery 108. If the cap 112 is to be used generally near sources of AC voltage, then the battery 108 can be eliminated. In addition to the AC power line cord 117, or in lieu thereof, the cap 112 can be equipped with a DC cord 118 connected to an AC/DC converter 119. The AC/DC converter 119 can be plugged into a standard AC outlet so that a resulting DC voltage is generated. The DC voltage is coupled to the circuits of the cap 112 via the DC cord 118. In either case, the cap 112 can be equipped with appropriate connectors for allowing easy connection/disconnection of either the AC power line cord 117 or the DC cord 118 to the respective electrical circuits of the cap 112. The wearer of the cap 112 can still move about while being treated by the light emitted from the light emitting devices 102.

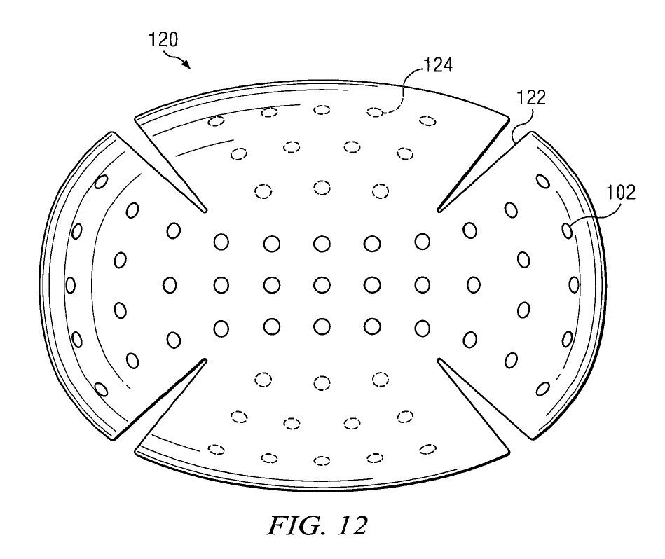

FIG. 12 illustrates another embodiment of a light emitting insert 120. The insert 120 is adapted for accommodating various head sizes. The size of the light emitting insert 120 can be changed by virtue of the slots, one shown as numeral 122. While four slots 122 are illustrated, other numbers of slots can be used. In all other respects, the light emitting insert 120 is constructed very much like that described above in connection with FIGS. 9a, 9b and 10. Since the plastic material from which the insert 120 is constructed is somewhat stiff, the insert 120 will spring outwardly and conform to the size of the cap into which it is inserted. It is contemplated that the general width of each slot 122 can be about one eighth inch to about one fourth inch. The light emitting devices 102 are located in an hourglass pattern, although this pattern can be other shapes. The insert 120 can be optionally equipped with light emitting devices 124 to illuminate the sides of the person's head, just above the ears.

While the various embodiments of the headgear according to the invention have been described in connection for use with a baseball type cap, this is not a limitation. Rather, the principles and concepts of the headgear of the invention can be used with a dress hat with a brim that encircles the crown piece, a helmet, a hairpiece or wig, a dew rag, any type of covering worn on a person's head, whether for purposes of protection from the weather or environment, for professional affiliations, sports, fun and novelty purposes, religious purposes, ceremonial purposes, cultural purposes, work and safety helmets, and any other type of device that can be worn on a person's head. In order to accommodate the light emitting headgear of the invention in a dress hat, for example, the shape of the light emitting headgear might have to be modified to fit the same, but that is well within the skill of those familiar with the art.

The foregoing describes the various embodiments in terms of use with a light emitting device, including laser diodes and LEDs. In every embodiment, the devices are preferably of the type that optimizes the regeneration or regrowth of hair. To that end, it is believed that light emitted in the wavelength range of about 400-1020 nm is well adapted for serving such purposes. A light wavelength of 650 nm is believed to be preferable. The power supplied to such devices is preferably five milliwatt, or less, to comply with the cosmetic use of the embodiments of the invention. Other power levels can be used which are consonant with FDA guidelines. The light emitted devices can emit a collimated beam of light, but preferably are constructed to emit an uncollimated beam of light to provide a wide area of coverage on the scalp of the user of the device. A light emitting device can be made with a lens cover over a collimated lens to diffuse the light beam and provide uncollimated rays of light, a well as create different shape patterns or lines. The light source employed can also be used with optical fibers and/or beam splitters to produce multiple beams of light. In every embodiment, the light emitting devices can be operated in a continuous mode, or pulsed at a predetermined rate. When operated in a pulsed mode, a miniature oscillator circuit can be employed to generate the timing of the pulses by which the light emitting devices are operated. The various features illustrated throughout the drawings can be utilized together to utilize the respective advantages of the such features.

While the preferred and other embodiments of the invention have been disclosed with reference to specific hair restoration headgear, and associated methods thereof, it is to be understood that many changes in detail may be made as a matter of engineering choices without departing from the spirit and scope of the invention, as defined by the appended claims.

What is claimed is:

1. Headgear for administering hair restoration treatments, comprising:
a headgear adapted for wearing by a person, said headgear constructed to appear as well known headgear worn by persons in public;
one or more light emitting devices attached to said headgear to illuminate a person's head when wearing said headgear; and
means for powering the light emitting devices so that when the headgear is worn by a user, the user can move about while administering the light to the wearer's head.

2. The headgear of claim 1, wherein said headgear comprises a baseball cap.

3. The headgear of claim 1, wherein said headgear comprises at least one of a dress hat, a casual hat, or a wig.

4. The headgear of claim 1, further including a switch attached to said headgear for controlling power delivered to said light emitting devices.

5. The headgear of claim 4, further including a timer responsive to actuation of said switch for providing power from said battery to the light emitting devices for a specified period of time.

6. The headgear of claim 1, further including a visual display for displaying a time of treatment.

7. The headgear of claim 6, wherein said timer provides an visual indication of a time remaining in which the light emitting devices will be powered by said battery.

8. The headgear of claim 4, wherein said headgear comprises a baseball cap, and said switch is attached to a bill of said cap.

9. The headgear of claim 1, wherein said light emitting devices are arranged in said headgear so as to illuminate only a desired shape of an area to be treated.

10. The headgear of claim 1, further including a pocket in said headgear for holding said battery.

11. The headgear of claim 1, further including a connector mounted to said headgear and connected to said battery so that a battery charger can be coupled to said connector.

12. The headgear of claim 1, wherein said light emitting devices comprise laser diodes.

13. The headgear of claim 1, further including a plurality of strips fastened in a crown portion of said headgear, and each strip having fastened thereto plural light emitting devices.

14. The headgear of claim 1, further including a dome-shaped member adapted for fitting within a crown portion of said headgear, and said light emitting devices are attached to said dome-shaped member.

15. Headgear for administering hair restoration treatments, comprising:
a baseball cap having a crown portion and a bill attached to said crown portion;
one or more light emitting devices attached to said baseball cap within said crown portion to illuminate a person's head when wearing said headgear; and
a battery for powering the light emitting devices, said battery attached to the baseball cap, whereby said baseball cap can be worn anywhere while administering the light to the wearer's head without the treatment being conspicuous.

16. The headgear of claim 15, further including plural strips supported within the crown portion of said baseball cap, each said strip including plural light emitting devices.

17. The headgear of claim 15, further including a switch attached to the bill of said baseball cap, said switch electrically connected to said battery and operable to apply battery power to said light emitting devices.

18. The headgear of claim 17, further including a timer responsive to actuation of said switch for allowing battery power to be applied to said light emitting devices a specified period of time.

19. A method of treating a person's hair with light energy, comprising:
arranging a plurality of light emitting devices in a headgear to be worn by the person;
constructing the headgear so that it can be placed on the person and the person can wear the headgear and move about with the headgear; and
applying power to said light emitting devices while the person wears said headgear and moves about.

20. The method of claim 19, further including fastening a visual display to the headgear to provide a visual indication of a time related to the treatment with the light energy.
